(19) United States
(12) Patent Application Publication
(10) Pub. No.: US 2025/0067742 A1
(43) Pub. Date: Feb. 27, 2025

(54) NONINVASIVE LIVER CANCER OCCURRENCE DETECTING METHOD

(71) Applicant: EpiSante Biomedical Co., Ltd., Taipei City (TW)

(72) Inventors: Chang-Yi LU, Hsinchu County (TW); Pey-Jey PENG, Hsinchu City (TW)

(21) Appl. No.: 18/814,308

(22) Filed: Aug. 23, 2024

Related U.S. Application Data
(60) Provisional application No. 63/578,525, filed on Aug. 24, 2023.

(51) Int. Cl. G01N 33/574

(52) U.S. Cl. CPC G01N 33/57438

(57) ABSTRACT

A noninvasive liver cancer occurrence detecting method has steps of performing biomarker identification of a group of differentially methylated genes in a computing system; performing quantitative measurement of the methylation levels of a plurality of biomarkers selected from the group of differentially methylated genes with quantitative methylation-specific PCR (qMSP) in the computing system; performing calculation in the computing system to obtain M-score of the selected biomarkers according to the measured methylation levels of the selected biomarkers; and performing a risk level evaluation of liver cancer with the M-score in the computing system.

### CROSS-REFERENCE TO RELATED APPLICATIONS

The present application claims the benefit of the U.S. provisional application Ser. No. 63/578,525, filed on Aug. 24, 2023, which is incorporated here by reference in its entirety.

### REFERENCE TO AN ELECTRONIC SEQUENCE LISTING

The content of the electronic sequence listing (2024-08-23_SequenceListing.xml; Size: 59 kb; and Date of Creation: Aug. 23, 2024) is herein incorporated by reference in its entirety.

### TECHNICAL FIELD

The present invention relates to a noninvasive liver cancer occurrence detecting method by using differentially methylated genes as biomarkers and measuring methylation levels of these biomarkers. The differentially methylated genes may be selected from a group consisting of the STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 gene. Moreover, a methylation prediction model developed for use in the noninvasive liver cancer occurrence detection is proposed.

### BACKGROUND

Hepatocellular carcinoma (HCC) is known as one of the most fatal cancers worldwide, causing 830,000 deaths every year. Due to rapid tumor progression, annual deaths from HCC nearly match the total number of cases, highlighting its high malignancy, poor prognosis, and high mortality. The incidence of HCC varies significantly by geographic location, likely due to regional differences in exposure to hepatitis B virus (HBV) and hepatitis C virus (HCV). Besides hepatitis virus infection, cirrhosis from any cause is a major risk factor for HCC.

HCC is relatively resistant to chemotherapy, with no chemotherapeutic agents shown to improve overall survival. Surgical interventions, such as partial liver resection and liver transplantation, are the only curative treatments for HCC. However, less than 30% of HCC patients are eligible for surgery, mainly due to late-stage diagnosis and multiple lesions on the cirrhotic or fibrotic liver. Therefore, early detection of HCC, when effective treatment can be applied, is crucial for improving overall survival.

Despite its unsatisfactory sensitivity and specificity, serum alpha-fetoprotein (AFP) is the most widely used tumor marker for HCC screening and surveillance. Using a cut-off value of 20 ng/ml, the reported sensitivities of AFP for HCC in cirrhotic patients range from 41% to 65% [1]. However, during the early stages of HCC progression, detection rates can be as low as one-third, as 80% of small HCC cases do not show elevated serum AFP levels [2,3]. By contrast, elevated AFP levels are also observed in other chronic liver diseases, such as cirrhosis and hepatic inflammation, as well as other types of cancer, including non-seminomatous germ cell tumors and gastrointestinal cancers [4]. Therefore, other biomarkers are needed to supplement AFP in order to improve the accuracy of diagnosis, especially for early HCC.

In recent decades, DNA methylation has increasingly been recognized as a valuable biomarker for the early detection and diagnosis of cancer. DNA methylation is a key mechanism that regulates gene expression in normal cells and plays a role in many physiological events. Aberrant DNA methylation can result in a variety of human diseases, including cancers. DNA methyltransferases catalyze DNA methylation by adding a methyl group to the carbon-5 position of cytosine residues in CpG dinucleotides. Methylation of the promoter or 5′ region of CpG islands can lead to transcriptional repression of downstream genes. There is mounting evidence that DNA hypermethylation can downregulate tumor suppressor and DNA repair genes, while hypomethylation can upregulate oncogenes during the early stages of carcinogenesis [5,6]. DNA methylation involves the covalent binding of a methyl group to genomic DNA, making it more stable than protein or RNA markers. Additionally, methylation markers can be detected in various types of liquid biopsies, such as blood, urine, saliva, and stool, providing a non-invasive method for monitoring cancer progression [7].

It is crucial to develop new methods for detecting liver cancer occurrence to improve early-stage detection rates. DNA methylation biomarkers have significant clinical value as markers for HCC diagnosis and prognosis. In our earlier study, we used a whole-genome approach to identify significant DNA methylation profiles from HCC cell lines and tissues [8-10].

The following is a list of references that are occasionally cited in the above-mentioned description. The disclosures of these references are incorporated by reference here in their entirety.
[1] Samir Gupta, Stephen Bent, Jeffrey Kohlwes. Test characteristics of alpha-fetoprotein for detecting hepatocellular carcinoma in patients with hepatitis C. A systematic review and critical analysis. Ann Intern Med. 2003 Jul. 1; 139 (1): 46-50. doi: 10.7326/0003-4819-139-1-200307010-00012.
[2] Jingjing Zong, Zhe Fan, Yewei Zhang. Serum Tumor Markers for Early Diagnosis of Primary Hepatocellular Carcinoma. J Hepatocell Carcinoma. 2020 Dec. 18; 7:413-422. doi: 10.2147/JHC.S272762.
[3] Basim Saleh Samman, Albadr Hussein, Razan Saleh Samman, Abdulaziz Saud Alharbi. Common Sensitive Diagnostic and Prognostic Markers in Hepatocellular Carcinoma and Their Clinical Significance: A Review. Cureus. 2022 Apr. 8; 14 (4): e23952. doi: 10.7759/cureus.23952.
[4] N Zamcheck, G Pusztaszeri. CEA, AFP and other potential tumor markers. CA Cancer J Clin. 1975 July-August; 25 (4): 204-14. doi: 10.3322/canjclin.25.4.204.
[5] Marina Bibikova, Bret Barnes, Chan Tsan, Vincent Ho, Brandy Klotzle, Jennie M Le, David Delano, Lu Zhang, Gary P Schroth, Kevin L Gunderson, Jian-Bing Fan, Richard Shen. High density DNA methylation array with single CpG site resolution. Genomics. 2011 October; 98 (4): 288-95. doi: 10.1016/j.ygeno.2011.07.007.
[6] Martin Widschwendter, Sophia Apostolidou, Elke Raum, Dietrich Rothenbacher, Heidi Fiegl, Usha Menon, Christa Stegmaier, Ian J Jacobs, Hermann Brenner. Epigenotyping in peripheral blood cell DNA and breast cancer risk: a proof of principle study. PLOS One. 2008 Jul. 16; 3 (7): e2656. doi: 10.1371/journal.pone.0002656.
[7] Huiyan Luo, Wei Wei, Ziyi Ye, Jiabo Zheng, Rui-Hua Xu. Liquid Biopsy of Methylation Biomarkers in Cell-Free DNA. Trends Mol Med. 2021 May; 27 (5): 482-500. doi: 10.1016/j.molmed.2020.12.011.
[8] Chang-Yi Lu, Sen-Yung Hsieh, Yen-Jung Lu, Chi-Sheng Wu, Lih-Chyang Chen, Shao-Jung Lo, Cheng-Tao Wu, Min-Yuan Chou, Tim Hui-Ming Huang, Yu-Sun Chang. Aberrant DNA methylation profile and frequent methylation of KLK10 and OXGR1 genes in hepatocellular carcinoma. Genes Chromosomes Cancer. 2009 December; 48 (12): 1057-68. doi: 10.1002/gcc.20708.
[9] Chang-Yi Lu, Kai-Yuan Lin, Meng-Tsung Tien, Cheng-Tao Wu, Yih-Huei Uen, Tzu-Ling Tseng. Frequent DNA methylation of MiR-129-2 and its potential clinical implication in hepatocellular carcinoma. Genes Chromosomes Cancer. 2013 July; 52 (7): 636-43. doi: 10.1002/gcc.22059.
[10] Chang-Yi Lu, Shih-Ya Chen, Hui-Ling Peng, Pu-Yeh Kan, Wan-Chi Chang, Chia-Jui Yen. Cell-free methylation markers with diagnostic and prognostic potential in hepatocellular carcinoma. Oncotarget. 2017 Jan. 24; 8 (4): 6406-6418. doi: 10.18632/oncotarget.14115.

### SUMMARY

The present invention provides a noninvasive method to detect and quantitatively measure the methylation levels of genes serving as biomarkers for liver cancer occurrence in a computing system. The biomarker evaluation of these methylated genes will complement the conventional prediction tools and facilitate more accurate detection of liver cancer occurrence.

The present disclosure provides a noninvasive liver cancer occurrence detecting method. In one embodiment, the method includes the following steps: (a) performing biomarker identification of a group of differentially methylated genes in a computing system equipped with a microprocessor and designed to process, analyze, simulate, and model biological data, by respectively detecting methylation levels of the differentially methylated genes, the group consists of STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 gene; (b) performing quantitative measurement of the methylation levels of a plurality of biomarkers selected from the group of the differentially methylated genes with quantitative methylation-specific PCR (qMSP) in the computing system, wherein the selected biomarkers are the STRIP1 gene, the PGAM2 gene, the AAMP gene, and the ZNF497 gene; (c) performing calculation using a formula M-score=X1+X2×ln(STRIP1)+X3×ln(PGAM2)+X4×ln(AAMP)+X5×ln(ZNF497) with a logistic regression analysis in the computing system to obtain M-score of the selected biomarkers according to the methylation levels of the selected biomarkers, wherein X1 ranges from 1.266 to 3.242, X2 ranges from 0.116 to 0.305, X3 ranges from 0.114 to 0.233, X4 ranges from 0.396 to 1.093, X5 ranges from 0.099 to 0.211, ln(STRIP1) represents a hyperbolic logarithm of the methylation level of the STRIP1 gene, ln(PGAM2) represents a hyperbolic logarithm of the methylation level of the PGAM2 gene, ln(AAMP) represents a hyperbolic logarithm of the methylation level of the AAMP gene, and ln(ZNF497) represents a hyperbolic logarithm of the methylation level of the ZNF497 gene; and (d) performing a risk level evaluation of liver cancer with the M-score of the selected biomarkers in the computing system.

In one embodiment, the noninvasive liver cancer occurrence detecting method further includes a step of performing a receiver operating characteristic (ROC) curve analysis of the M-score of the selected biomarkers in the computing system.

In one embodiment, wherein the risk of being afflicted with liver cancer increases with the M-score compared to a reference value.

In one embodiment, the step of performing the quantitative measurement of the methylation levels of the selected biomarkers with the quantitative methylation-specific PCR (qMSP) in the computing system includes performing a calculation using a formula 2^{[Ct(housekeeping gene)-Ct(biomarker)]}×100 based on a difference in Ct value between each of the selected biomarkers and a housekeeping gene, the Ct value is of single data points derived from real-time PCR amplification plots.

In one embodiment, the mentioned housekeeping gene is a gene selected from a group consisting of β-actin, GAPDH, HPRT, YWHAZ, ARBP, SDHA and UBC.

In one embodiment, the step of performing the quantitative measurement of the methylation levels of the selected biomarkers with the quantitative methylation-specific PCR (qMSP) in the computing system includes using a kit to detect, the kit has a plurality of primer and probe sets targeting the STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 genes, respectively, wherein each of the primer and probe sets includes a primer-pair and a probe; and a qPCR master mix having Taq DNA polymerase, dNTPs, MgCl2 and buffer.

In one embodiment, each of the mentioned primer-pair includes a sense primer and an antisense primer, and wherein the sense primer, the antisense primer, and the probe separately have a sequence associated with the correspondingly targeted gene.

In one embodiment, the methylation level of the STRIP1 gene is calculated using formula 2^{[Ct(β-actin)-Ct(STRIP1)]}×100, the methylation level of the PGAM2 gene is calculated using formula 2^{[Ct(β-actin)-Ct(PGAM2)]}×100, the methylation level of the AAMP gene is calculated using formula 2^{[Ct(β-actin)-Ct(AAMP)]}×100, and the methylation level of the ZNF497 gene is calculated using formula 2^{[Ct(β-actin)-Ct(ZNF497)]}×100.

In another aspect, a kit may be used in the noninvasive liver cancer occurrence detecting method. The kit has a plurality of primer-pair and probe sets to separately detect the methylation level of the STRIP1 gene, the ZNF649 gene, the CFAP57 gene, the GGTA1 gene, the PGAM2 gene, the FRMD4A gene, the GJB6 gene, the AAMP gene, the miR-203A, the SLC22A2 gene, the BCAS3 gene, the TSPYL5 gene, and the ZNF497 gene. Each of the primer-pair and probe sets has a primer-pair and a probe.

Alternatively, a kit having a plurality of primer-pair and probe sets to separately detect the methylation level of the STRIP1 gene, the PGAM2 gene, the AAMP gene, and the ZNF497 gene may be used.

Biomarkers regulated by DNA methylation are identified in a computing system as participating in the liver cancer occurrence and may potentially function as a powerful predictor for liver cancer. The “M-score” calculated in a computing manner in the present invention has proven to be effective in predicting liver cancer patients. The M-score exceeds the predictive capabilities of serum markers.

This invention discloses the role of DNA methylation in predicting the occurrence of live cancer. Some of the biomarkers that are differentially methylated between liver cancer patients and non-tumor control may be used to calculate the “M-score” for liver cancer diagnosis. This invention compares M-score with other clinical factors, such as AFP, and proves that M-score had a better performance in predicting the liver cancer occurrence.

Various other objects, advantages, and features of the present invention will become readily apparent from the ensuing detailed description accompanying drawings, and the novel features will be particularly pointed out in the appended claims.

### BRIEF DESCRIPTION OF DRAWINGS

The following detailed descriptions, given by way of example, and not intended to limit the present invention solely thereto, will be best be understood in conjunction with the accompanying figures.

FIG. 1A indicates the methylation level of the STRIP1 gene in bio-samples coming from HCC (liver cancer) subjects and healthy (non-tumor control) donors.

FIG. 1B indicates the methylation level of the ZNF649 gene in bio-samples coming from HCC (liver cancer) subjects and healthy (non-tumor control) donors.

FIG. 1C indicates the methylation level of the CFAP57 gene in bio-samples coming from HCC (liver cancer) subjects and healthy (non-tumor control) donors.

FIG. 1D indicates the methylation level of the GGTA1 gene in bio-samples coming from HCC (liver cancer) subjects and healthy (non-tumor control) donors.

FIG. 1E indicates the methylation level of the PGAM2 gene in bio-samples coming from HCC (liver cancer) subjects and healthy (non-tumor control) donors.

FIG. 1F indicates the methylation level of the FRMD4A gene in bio-samples coming from HCC (liver cancer) subjects and healthy (non-tumor control) donors.

FIG. 1G indicates the methylation level of the GJB6 gene in bio-samples coming from HCC (liver cancer) subjects and healthy (non-tumor control) donors.

FIG. 1H indicates the methylation level of the AAMP gene in bio-samples coming from HCC (liver cancer) subjects and healthy (non-tumor control) donors.

FIG. 1I indicates the methylation level of the miR-203A in bio-samples coming from HCC (liver cancer) subjects and healthy (non-tumor control) donors.

FIG. 1J indicates the methylation level of the SLC22A2 gene in bio-samples coming from HCC (liver cancer) subjects and healthy (non-tumor control) donors.

FIG. 1K indicates the methylation level of the BCAS3 gene in bio-samples coming from HCC (liver cancer) subjects and healthy (non-tumor control) donors.

FIG. 1L indicates the methylation level of the TSPYL5 gene in bio-samples coming from HCC (liver cancer) subjects and healthy (non-tumor control) donors.

FIG. 1M indicates the methylation level of the ZNF497 gene in bio-samples coming from HCC (liver cancer) subjects and healthy (non-tumor control) donors.

FIG. 2A illustrates receiver operator characteristic (ROC) curves for the M-score of the STRIP1 gene, PGAM2 gene, AAMP gene, and ZNF497 gene in the receiver operating characteristic curve (ROC) analysis.

FIG. 2B illustrates receiver operator characteristic (ROC) curves for AFP in the receiver operating characteristic curve (ROC) analysis.

FIG. 3 is a flow chart illustrating a noninvasive liver cancer occurrence detecting method according to one embodiment of the present invention.

### DETAILED DESCRIPTION OF THE EMBODIMENTS

In the following description, the biomarker/biomarkers, and the corresponding embodiments of the detection/validation/identification/quantification methods are set forth as preferred examples. It will be clear to those skilled in the art that modifications, including additions and/or substitutions, may be made without departing from the scope and spirit of the invention. Specific details may be omitted so as not to obscure the invention; however, the disclosure is written to enable one skilled in the art to practice the teachings here without undue experimentation.

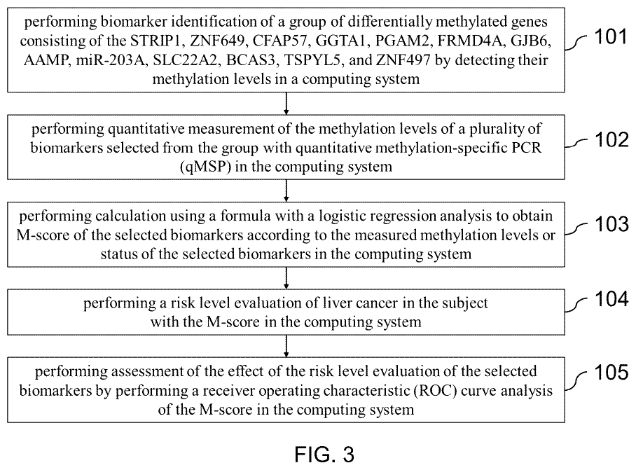

In one embodiment, a noninvasive liver cancer occurrence detecting method used to evaluate the risk of suffering liver cancer in a subject may include, but is not limited to, the following steps 101 to steps 104, as shown in FIG. 3. The subject may include, but is not limited to, a mammal, such as a human, ape, monkey, cat, dog, rabbit, guinea pig, rat, or mouse. In one embodiment, the subject is a human.

Step 101: performing biomarker identification of a group of differentially methylated genes consisting of the STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 gene by detecting the methylation levels or status of these differentially methylated genes, respectively, in bio-samples coming from the subject. The bio-sample may include, but is not limited to, blood, plasma, serum, liver tissue, saliva, sputum, semen, intestinal digestive, respiratory lavage, and feces. In one embodiment, the bio-sample is plasma or serum. The above-mentioned genes are used as potential biomarkers for liver cancer occurrence in the present invention.

The step 101 is preferably performed in a computing system, such as a computer, or a system equipped with a microprocessor capable of computing data and processing bio-signals. The computing system is designed to process, analyze, simulate, and model biological data and may employ specialized algorithms, software, and high-performance hardware to study and solve complex problems in biology, bioinformatics, computational biology, and other life science domains. Common applications of the computing system include genome sequencing and assembly, molecular modeling and simulation, bioinformatics databases, gene expression analysis, protein structure prediction, and drug design. Key components that enable the computing system to manage big biological data include clustering algorithms, artificial intelligence/machine learning models, distributed storage, and cloud computing, as well as advanced visualization capabilities. In one example, the computing system may include a biomaterial input device, a biomaterial processing unit, a bio-signal detection unit, and a data processing unit incorporating a microprocessor.

Step 102: performing quantitative measurement of the methylation levels of a plurality of biomarkers selected from the group of differentially methylated genes with quantitative methylation-specific PCR (qMSP) in the computing system. In one example, the selected biomarkers are the STRIP1, PGAM2, AAMP, and ZNF497 genes.

Step 103: performing calculation using a formula with a logistic regression analysis in the computing system to obtain M-score of the selected biomarkers according to the measured methylation levels or status of the selected biomarkers. In one example, the selected biomarkers are the STRIP1, PGAM2, AAMP, and ZNF497 genes.

Step 104: performing a risk level evaluation of liver cancer in the subject with the M-score in the computing system. In one embodiment, the subject has the risk of afflicting with liver cancer when the M-score is higher compared to a pre-confirmed reference value.

In one embodiment, the noninvasive liver cancer occurrence detecting method used to evaluate the risk of suffering liver cancer in the subject may further include the following step, as shown in FIG. 3.

Step 105: performing assessment of the effect of the risk level evaluation of the selected biomarkers by performing a receiver operating characteristic (ROC) curve analysis of the M-score in the computing system.

The step 101 of detecting the methylation levels or status of the STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 gene may utilize analysis methods include, but is not limited to, quantitative methylation-specific polymerase chain reaction (quantitative methylation-specific PCR, qMSP), combined bisulfite restriction analysis (COBRA), Bisulfite Sequencing, Pyrosequencing, Next Generation sequencing (NGS), and DNA Methylation Array Chip Analysis. The analysis methods are performed in the computing system.

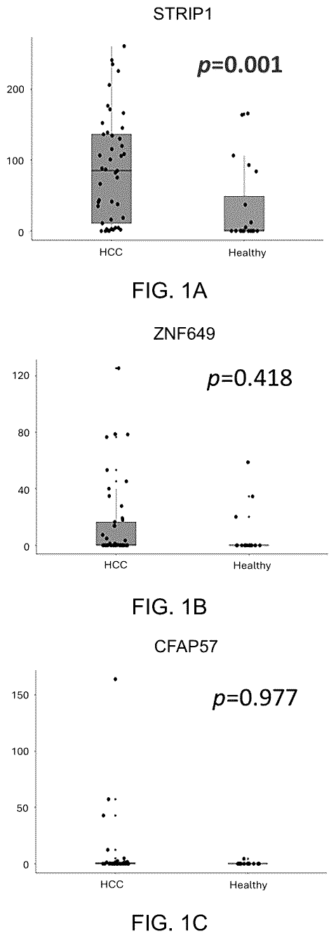

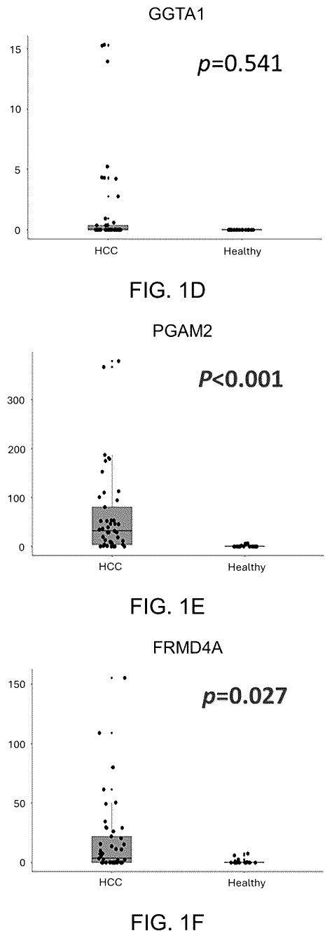

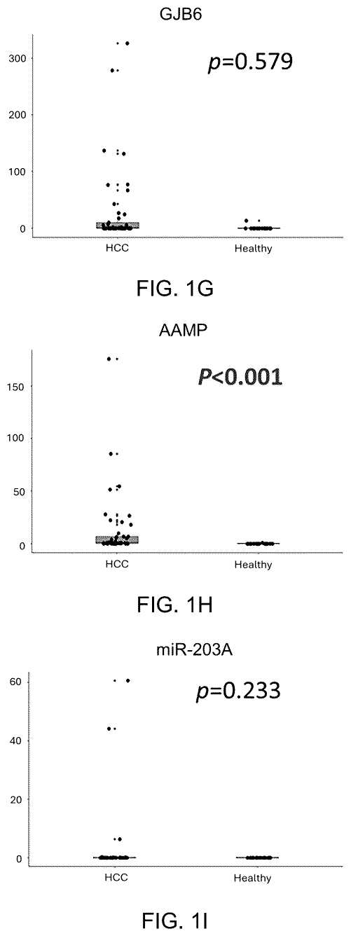

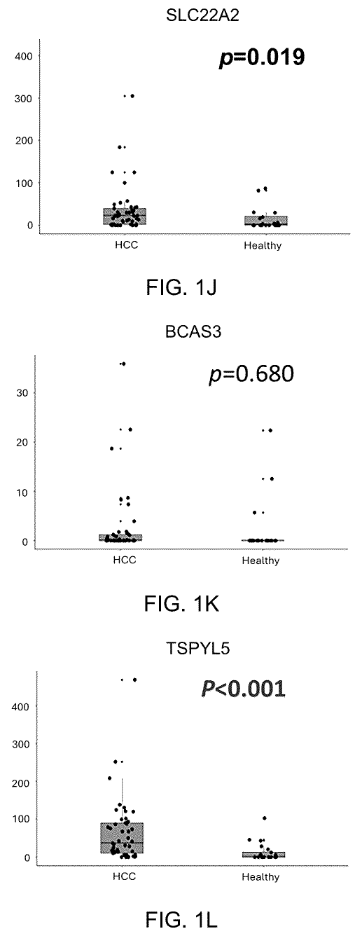

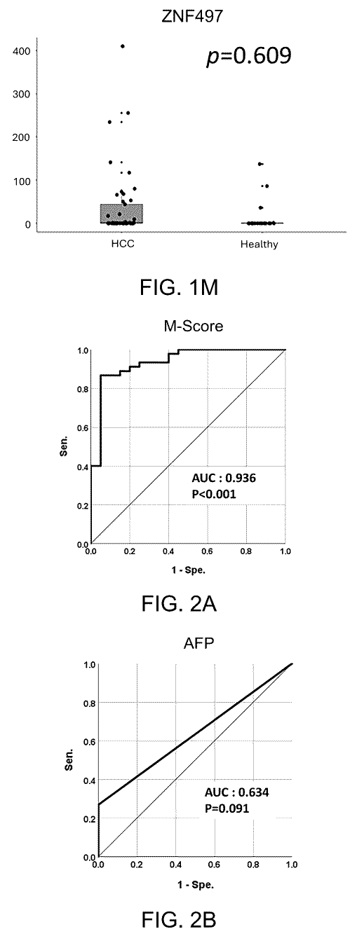

In one embodiment, the step 101 of detecting the methylation levels or status of the STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 gene utilizes the quantitative methylation-specific PCR. In one example, the bio-samples are 65 plasma samples coming from 20 healthy donors and 45 HCC patients. Referring to FIG. 1A to 1M, the STRIP1, PGAM2, FRMD4A, AAMP, SLC22A2, and TSPYL5 genes showed higher methylation level in HCC patients than in healthy donors, with statistically significant differences, i.e., the P value is less than 0.05. On the other hand, as shown in Table 1, the methylation status of three candidates of the potential biomarkers, including the ZNF649 gene, GGTA1 gene, and GJB6 gene, were found to be significantly correlated with HCC occurrence in statistics, i.e., the P value is less than 0.05. As a result, the STRIP1, PGAM2, FRMD4A, AAMP, SLC22A2, TSPYL5, ZNF649, GGTA1, and GJB6 genes may be considered as biomarkers for HCC diagnosis.

TABLE 1

| Candidates | Subjects | Unmethylation | Methylation | P value |
|---|---|---|---|---|
| ZNF649 | Healthy | 17 | 3 | 0.032298 |
| | HCC | 26 | 19 | |
| CFAP57 | Healthy | 19 | 1 | 0.087428 |
| | HCC | 35 | 10 | |
| GGTA1 | Healthy | 20 | 0 | 0.044052 |
| | HCC | 37 | 8 | |
| GJB6 | Healthy | 19 | 1 | 0.006417 |
| | HCC | 28 | 17 | |
| miR-203A | Healthy | 20 | 0 | 0.086526 |
| | HCC | 39 | 6 | |
| BCAS3 | Healthy | 17 | 3 | 0.230240 |
| | HCC | 32 | 13 | |
| ZNF497 | Healthy | 17 | 3 | 0.092625 |
| | HCC | 29 | 16 | |

In one embodiment, the detection of the methylation levels or status of the STRIP1 gene may use a primer-pair including a sense primer, and an antisense primer, and a probe, wherein the sense primer has a sequence of at least 85% sequence similarity to SEQ ID NO: 1 as listed in Table 2, the antisense primer has a sequence of at least 85% sequence similarity to SEQ ID NO: 2 as listed in Table 2, and the probe has a sequence of at least 85% sequence similarity to SEQ ID NO: 3 as listed in Table 2. In one embodiment, the detection of the methylation level or status of the ZNF649 gene may use a primer-pair including a sense primer, and an antisense primer, and a probe, wherein the sense primer has a sequence of at least 85% sequence similarity to SEQ ID NO: 4 as listed in Table 2, the antisense primer has a sequence of at least 85% sequence similarity to SEQ ID NO: 5 as listed in Table 2, and the probe has a sequence of at least 85% sequence similarity to SEQ ID NO: 6 as listed in Table 2. In one embodiment, the detection of the methylation level or status of the CFAP57 gene may use a primer-pair including a sense primer, and an antisense primer, and a probe, wherein the sense primer has a sequence of at least 85% sequence similarity to SEQ ID NO: 7 as listed in Table 2, the antisense primer has a sequence of at least 85% sequence similarity to SEQ ID NO: 8 as listed in Table 2, and the probe has a sequence of at least 85% sequence similarity to SEQ ID NO: 9 as listed in Table 2. In one embodiment, the detection of the methylation level or status of the GGTA1 gene may use a primer-pair including a sense primer, and an antisense primer, and a probe, wherein the sense primer has a sequence of at least 85% sequence similarity to SEQ ID NO: 10 as listed in Table 2, the antisense primer has a sequence of at least 85% sequence similarity to SEQ ID NO: 11 as listed in Table 2, and the probe has a sequence of at least 85% sequence similarity to SEQ ID NO: 12 as listed in Table 2. In one embodiment, the detection of the methylation level or status of the PGAM2 gene may use a primer-pair including a sense primer, and an antisense primer, and a probe, wherein the sense primer has a sequence of at least 85% sequence similarity to SEQ ID NO: 13 as listed in Table 2, the antisense primer has a sequence of at least 85% sequence similarity to SEQ ID NO: 14 as listed in Table 2, and the probe has a sequence of at least 85% sequence similarity to SEQ ID NO: 15 as listed in Table 2. In one embodiment, the detection of the methylation level or status of the FRMD4A gene may use a primer-pair including a sense primer, and an antisense primer, and a probe, wherein the sense primer has a sequence of at least 85% sequence similarity to SEQ ID NO: 16 as listed in Table 2, the antisense primer has a sequence of at least 85% sequence similarity to SEQ ID NO: 17 as listed in Table 2, and the probe has a sequence of at least 85% sequence similarity to SEQ ID NO: 18 as listed in Table 2. In one embodiment, the detection of the methylation level or status of the GJB6 gene may use a primer-pair including a sense primer, and an antisense primer, and a probe, wherein the sense primer has a sequence of at least 85% sequence similarity to SEQ ID NO: 19 as listed in Table 2, the antisense primer has a sequence of at least 85% sequence similarity to SEQ ID NO: 20 as listed in Table 2, and the probe has a sequence of at least 85% sequence similarity to SEQ ID NO: 21 as listed in Table 2. In one embodiment, the detection of the methylation level or status of the AAMP gene may use a primer-pair including a sense primer, and an antisense primer, and a probe, wherein the sense primer has a sequence of at least 85% sequence similarity to SEQ ID NO: 22 as listed in Table 2, the antisense primer has a sequence of at least 85% sequence similarity to SEQ ID NO: 23 as listed in Table 2, and the probe has a sequence of at least 85% sequence similarity to SEQ ID NO: 24 as listed in Table 2. In one embodiment, the detection of the methylation level or status of the miR-203A may use a primer-pair including a sense primer, and an antisense primer, and a probe, wherein the sense primer has a sequence of at least 85% sequence similarity to SEQ ID NO: 25 as listed in Table 2, the antisense primer has a sequence of at least 85% sequence similarity to SEQ ID NO: 26 as listed in Table 2, and the probe has a sequence of at least 85% sequence similarity to SEQ ID NO: 27 as listed in Table 2. In one embodiment, the detection of the methylation level or status of the SLC22A2 gene may use a primer-pair including a sense primer, and an antisense primer, and a probe, wherein the sense primer has a sequence of at least 85% sequence similarity to SEQ ID NO: 28 as listed in Table 2, the antisense primer has a sequence of at least 85% sequence similarity to SEQ ID NO: 29 as listed in Table 2, and the probe has a sequence of at least 85% sequence similarity to SEQ ID NO: 30 as listed in Table 2. In one embodiment, the detection of the methylation level or status of the BCAS3 gene may use a primer-pair including a sense primer, and an antisense primer, and a probe, wherein the sense primer has a sequence of at least 85% sequence similarity to SEQ ID NO: 31 as listed in Table 2, the antisense primer has a sequence of at least 85% sequence similarity to SEQ ID NO: 32 as listed in Table 2, and the probe has a sequence of at least 85% sequence similarity to SEQ ID NO: 33 as listed in Table 2. In one embodiment, the detection of the methylation level or status of the TSPYL5 gene may use a primer-pair including a sense primer, and an antisense primer, and a probe, wherein the sense primer has a sequence of at least 85% sequence similarity to SEQ ID NO: 34 as listed in Table 2, the antisense primer has a sequence of at least 85% sequence similarity to SEQ ID NO: 35 as listed in Table 2, and the probe has a sequence of at least 85% sequence similarity to SEQ ID NO: 36 as listed in Table 2. In one embodiment, the detection of the methylation level or status of the ZNF497 gene may use a primer-pair including a sense primer, and an antisense primer, and a probe, wherein the sense primer has a sequence of at least 85% sequence similarity to SEQ ID NO: 37 as listed in Table 2, the antisense primer has a sequence of at least 85% sequence similarity to SEQ ID NO: 38 as listed in Table 2, and the probe has a sequence of at least 85% sequence similarity to SEQ ID NO: 39 as listed in Table 2.

In one embodiment, the STRIP1, PGAM2, AAMP, and ZNF497 genes are chosen as the selected biomarkers in the step 102. Moreover, a primer-pair including a sense primer and an antisense primer, and a probe correspondingly listed in Table 2 for the STRIP1, PGAM2, AAMP, and ZNF497 genes are used in the detection.

The usage of the quantitative methylation-specific PCR and the primer-pair to quantitatively measure the methylation levels or status of a methylated gene are described in the following examples.

In one example, a cell-free DNA from 400 μl plasma of a subject was bisulfite-converted by treatment of sodium bisulfite. The bisulfite-converted cell-free DNA was amplified by real-time quantitative methylation-specific PCR (qMSP) using fluorescent probes. Each reaction involved 1×qPCR Master Mix, 0.5 UM of each primer and 0.25 μM of probe in a total volume of 20 μl. Amplification was performed on the StepOnePlus Real-Time PCR System (Thermo Fisher Scientific). The methylation level of a selected biomarker of a methylated gene may be calculated from the formula: 2^{[Ct(housekeeping gene)-Ct(biomarker)]}×100, wherein Ct is the value of single data points derived from real-time PCR amplification plots, and the housekeeping gene may be a gene selected from a group consisting of β-actin, GAPDH, HPRT, YWHAZ, ARBP, SDHA and UBC. In one example, the methylation level was calculated as the difference in Ct value between beta-actin and the selected biomarkers using the following formula: 2^{[Ct(beta-actin)-Ct(biomarkers)]}×100. In one example, the primer-pairs and probe utilized in detecting methylation levels of the STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 gene are illustrated as follows.

TABLE 2

| Target | Primer/ | |
|---|---|---|
| gene | Probe | Sequence |
| STRIP1 | Sense | GGTAGYGTTCGGTCGAKAATTG |
| | primer | (SEQ ID No. 1) |
| | Antisense | CTCCCGAACGYCGAAARTAAA |
| | primer | (SEQ ID No. 2) |
| | Probe | AKGAGTAGGAAGCGASGTTGGTAGT |
| | | (SEQ ID No. 3) |
| ZNF649 | Sense | GGAATCSGAAGCGAGSTTTA |
| | primer | (SEQ ID No. 4) |
| | Antisense | ACCAATCCRAACGACCATRC |
| | primer | (SEQ ID No. 5) |
| | Probe | MGTGAGATTKGTTATTATTCGGCGT |
| | | (SEQ ID No. 6) |
| CFAP57 | Sense | GGGTCGTTTCGTASGGATTR |
| | primer | (SEQ ID No. 7) |
| | Antisense | CTCSTCCTTCACSTCATTACAC |
| | primer | (SEQ ID No. 8) |
| | Probe | AAGTATTGCGGRTTGGGTTTGGCG |
| | | (SEQ ID No. 9) |
| GGTA1 | Sense | GGTTGAGGTAGKCGGATTATT |
| | primer | (SEQ ID No. 10) |
| | Antisense | CTCAYCCTCCCACGTAACTYAA |
| | primer | (SEQ ID No. 11) |
| | Probe | AATTAGTTKGGCGTGGTGGTYCGT |
| | | (SEQ ID No. 12) |
| PGAM2 | Sense | TTTGGACGGTACYGATTAGATG |
| | primer | (SEQ ID No. 13) |
| | Antisense | CCGTACTTAACGACMGTTTCT |
| | primer | (SEQ ID No. 14) |
| | Probe | TGTTRGTGGTGCGTATTTGGCGTT |
| | | (SEQ ID No. 15) |
| FRMD4A | Sense | GGMGAGAGAGGTAGAGTGTGTT |
| | primer | (SEQ ID No. 16) |
| | Antisense | AACKAACTCAACTCCCGATAAA |
| | primer | (SEQ ID No. 17) |
| | Probe | TGCGGCGTATARGTGGTATAGTTAGGGA |
| | | (SEQ ID No. 18) |
| GJB6 | Sense | GGTATCGGATTAGAGTCGGTMGA |
| | primer | (SEQ ID No. 19) |
| | Antisense | RGAAACCGAAACGCGTAAAC |
| | primer | (SEQ ID No. 20) |
| | Probe | TACGACCAAACACCCATCCCGYC |
| | | (SEQ ID No. 21) |
| AAMP | Sense | TGAGGAMGAAGAGGAGGAAGA |
| | primer | (SEQ ID No. 22) |
| | Antisense | CTATCGTCGAAACSCTCCATAC |
| | primer | (SEQ ID No. 23) |
| | Probe | AAYCCCAACCCTCTTCGTTACCC |
| | | (SEQ ID No. 24) |
| miR-203A | Sense | CSCGGTTTGGAGTTAGAGTTATAG |
| | primer | (SEQ ID No. 25) |
| | Antisense | TACGAAACYCGACGCATAAA |
| | primer | (SEQ ID No. 26) |
| | Probe | ACGCGAAACMGATCCGTAAACTCC |
| | | (SEQ ID No. 27) |
| SLC22A2 | Sense | GGGTGATTATCGTTGTTATRGAAGG |
| | primer | (SEQ ID No. 28) |
| | Antisense | AATTTCCCACATAACCTCTACSC |
| | primer | (SEQ ID No. 29) |
| | Probe | TSGAGAATGTCGATGATGGGTAGTGG |
| | | (SEQ ID No. 30) |
| BCAS3 | Sense | GGTATGGTGGTACGTGTTRGTA |
| | primer | (SEQ ID No. 31) |
| | Antisense | CTCGCTCTATCACCCAAWCTAAA |
| | primer | (SEQ ID No. 32) |
| | Probe | TCGGGAGGTAGAGGTRGTAGTGAGTT |
| | | (SEQ ID No. 33) |
| TSPYL5 | Sense | GGAGSGTTTRGTCGTMGATATT |
| | primer | (SEQ ID No. 34) |
| | Antisense | AAACCACSCCGATTTCSA |
| | primer | (SEQ ID No. 35) |
| | Probe | TTKCGTGGGAATAGCGSGAATMGT |
| | | (SEQ ID No. 36) |
| ZNF497 | Sense | CGAGGCGGGRGGATTATTT |
| | primer | (SEQ ID No. 37) |
| | Antisense | TCKCGATCTCCTAACCTCGTAAT |
| | primer | (SEQ ID No. 38) |
| | Probe | TTTATCGGTCGSGTGTCGTGGTTT |
| | | (SEQ ID No. 39) |
| ß-actin | Sense | GGTGGAGGTAGTTAGGGTTTATTTGTA |
| | primer | (SEQ ID No. 40) |
| | Antisense | CCACACCACAAAATCACACTTAACCTCATTT |
| | primer | (SEQ ID No. 41) |
| | Probe | CACTTTTATTCAACTAATCTC |
| | | (SEQ ID No. 42) |

In one embodiment, the step 103 is performed to obtain the M-score of the selected biomarkers according to the methylation levels or status of the selected biomarkers being the STRIP1 gene, PGAM2 gene, AAMP gene, and ZNF497 gene by calculating the following formula: M-score=X1+X2×ln(STRIP1)+X3×ln(PGAM2)+X4×ln(AAMP)+X5×ln(ZNF497).

In the above formula, X1 ranges from 1.266 to 3.242, X2 ranges from 0.116 to 0.305, X3 ranges from 0.114 to 0.233, X4 ranges from 0.396 to 1.093, and X5 ranges from 0.099 to 0.211. In the above formula, the “ln(STRIP1)” represents a hyperbolic logarithm of the methylation level of the STRIP1 gene, and the methylation level of the STRIP1 gene is calculated using the formula: 2^{[(Ct(β-actin)-Ct(STRIP1)]}×100. In the above formula, the “ln(PGAM2)” represents a hyperbolic logarithm of the methylation level of the PGAM2 gene, and the methylation level of the PGAM2 gene is calculated using the formula: 2^{[(Ct(β-actin)-Ct(PGAM2)]}×100. In the above formula, the “ln(AAMP)” represents a hyperbolic logarithm of the methylation level of the AAMP gene, and the methylation level of the AAMP gene is calculated using the formula: 2^{[Ct(β-actin)-Ct(AAMP)]}×100. In the above formula, the “ln(ZNF497)” represents a hyperbolic logarithm of the methylation level of the ZNF497 gene, and the methylation level of the ZNF497 gene is calculated using the formula: 2^{[Ct(β-actin)-Ct(ZNF497)]}×100.

In one embodiment, the step 104 is performed with the M-score which is calculated according to the methylation levels of the STRIP1 gene, PGAM2 gene, AAMP gene and ZNF497 gene. In one example, the subject has the risk of afflicting with liver cancer when the M-score is higher compared to a pre-confirmed reference value. In other words, the risk of being afflicted with liver cancer increases with the M-score compared to the pre-confirmed reference value.

In one embodiment, the step 104 may include a step of determining the pre-confirmed reference value by respectively comparing the methylation level of the STRIP1 gene, PGAM2 gene, AAMP gene and ZNF497 gene in one group of the subjects known not to have liver cancer with another group known to have liver cancer and obtaining a cutoff value from the receiver operating characteristic (ROC) curves in the receiver operating characteristic analysis performed in the computing system. In one example, the pre-confirmed reference value is 0.54 and the subject has the risk of afflicting with liver cancer when the M-score is higher than 0.54.

Given the above, in one embodiment, a kit made of a plurality of primer and probe sets of methylation biomarkers and a qPCR master mix may be preferably used to detect the biomarker methylation in the liver cancer occurrence detection, wherein the primer and probe sets may respectively target the STRIP1 gene (SEQ ID No.1-3 of Table 2), PGAM2 gene (SEQ ID No.13-15 of Table 2), AAMP gene (SEQ ID No.22-24 of Table 2), ZNF497 gene (SEQ ID No.37-39 of Table 2), and β-actin gene (SEQ ID No.40-42 of Table 2), and the qPCR master mix may have Taq DNA polymerase, dNTPs, MgCl2 and buffer.

In one embodiment, the step 105 carries out a receiver operating characteristic (ROC) curve analysis to assess the performance or the diagnostic effect of the methylation biomarkers including the STRIP1 gene, PGAM2 gene, AAMP gene, and ZNF497 gene.

FIG. 2A illustrates receiver operator characteristic (ROC) curves for the M-score of the biomarkers including the STRIP1 gene, PGAM2 gene, AAMP gene, and ZNF497 gene. FIG. 2B illustrates receiver operator characteristic (ROC) curves for AFP (alpha-fetoprotein, cut-off at 20 ng/ml). The AUC (area under the curve) in view of the M-score in FIG. 2A is 0.936 (95% CI: 0.869-1, P<0.001), while the AUC in view of AFP in FIG. 2B is 0.634 (95% CI: 0.495-0.773, P=0.091). These P values show statistical significance for the M-score. In FIGS. 2A and 2B, the “1-Spe.” means “1-Specificity” and the “Sen.” means “Sensitivity.”

As shown in Table 3, the receiver operating characteristic (ROC) analysis determines the sensitivity, specificity, precision, false positive rate, false negative rate, and accuracy for the M-score and AFP, respectively. For the M-score, these values are 91.11%, 80.00%, 91.11%, 20.00%, 8.89%, and 87.69%, respectively. For AFP, these values are 26.83%, 100.00%, 100.00%, 0.00%, 73.17%, and 50.82%, respectively. These results shown that the performance of the M-score is superior to AFP in liver cancer occurrence detection.

TABLE 3

| characteristics | M-score | AFP |
|---|---|---|
| Sensitivity | 91.11% | 26.83% |
| Specificity | 80.00% | 100.00% |
| Precision | 91.11% | 100.00% |
| False Positive Rate | 20.00% | 0.00% |
| False Negative Rate | 8.89% | 73.17% |
| Accuracy | 87.69% | 50.82% |

Biomarkers regulated by DNA methylation were identified in a computing system as participating in the liver cancer occurrence and may potentially function as a powerful predictor for liver cancer. The “M-score” calculated in a computing manner in the present invention has proven to be effective in predicting liver cancer patients. The M-score exceeds the predictive capabilities of serum markers.

Having described at least one of the embodiments of the claimed invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure covers modifications and variations of this disclosure provided they come within the scope of the appended claims and their equivalents. Specifically, one or more limitations recited throughout the specification can be combined in any level of details to the extent they are described to carry out the noninvasive liver cancer occurrence detecting method.

What is claimed is:

1. A noninvasive liver cancer occurrence detecting method, comprising:
performing biomarker identification of a group of differentially methylated genes in a computing system designed to process, analyze, simulate, and model biological data and equipped with a microprocessor by respectively detecting methylation levels of the differentially methylated genes, the group consisting of STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 gene;
performing quantitative measurement of the methylation levels of a plurality of biomarkers selected from the group of the differentially methylated genes with quantitative methylation-specific PCR (qMSP) in the computing system, wherein the selected biomarkers are the STRIP1 gene, the PGAM2 gene, the AAMP gene, and the ZNF497 gene;
performing calculation using a formula M-score=X1+X2×ln(STRIP1)+X3×ln(PGAM2)+X4×ln(AAMP)+X5×ln(ZNF497) with a logistic regression analysis in the computing system to obtain M-score of the selected biomarkers according to the measured methylation levels of the selected biomarkers, wherein X1 ranges from 1.266 to 3.242, X2 ranges from 0.116 to 0.305, X3 ranges from 0.114 to 0.233, X4 ranges from 0.396 to 1.093, X5 ranges from 0.099 to 0.211, ln(STRIP1) represents a hyperbolic logarithm of the methylation level of the STRIP1 gene, ln(PGAM2) represents a hyperbolic logarithm of the methylation level of the PGAM2 gene, ln(AAMP) represents a hyperbolic logarithm of the methylation level of the AAMP gene, and ln(ZNF497) represents a hyperbolic logarithm of the methylation level of the ZNF497 gene; and
performing a risk level evaluation of liver cancer with the M-score of the selected biomarkers in the computing system.

2. The method of claim 1, further comprising:
performing a receiver operating characteristic (ROC) curve analysis of the M-score of the selected biomarkers in the computing system.

3. The method of claim 1, wherein the risk of being afflicted with liver cancer increases with the M-score compared to a reference value.

4. The method of claim 1, wherein the step of performing the quantitative measurement of the methylation levels of the selected biomarkers with the quantitative methylation-specific PCR (qMSP) in the computing system comprises performing a calculation using a formula 2^{[Ct(housekeeping gene)-Ct(biomarker)]}×100 based on a difference in Ct value between each of the selected biomarkers and a housekeeping gene, the Ct value is of single data points derived from real-time PCR amplification plots.

5. The method of claim 4, wherein the housekeeping gene is a gene selected from a group consisting of β-actin, GAPDH, HPRT, YWHAZ, ARBP, SDHA and UBC.

6. The method of claim 1, wherein the step of performing the quantitative measurement of the methylation levels of the selected biomarkers with the quantitative methylation-specific PCR (qMSP) in the computing system comprises using a kit to detect, the kit comprising:
a plurality of primer and probe sets targeting the STRIP1 gene, ZNF649 gene, CFAP57 gene, GGTA1 gene, PGAM2 gene, FRMD4A gene, GJB6 gene, AAMP gene, miR-203A, SLC22A2 gene, BCAS3 gene, TSPYL5 gene, and ZNF497 gene, respectively, wherein each of the primer and probe sets includes a primer-pair and a probe; and
a qPCR master mix having Taq DNA polymerase, dNTPs, MgCl2 and buffer.

7. The method of claim 6, wherein each of the primer-pair includes a sense primer and an antisense primer, and wherein the sense primer, the antisense primer, and the probe separately have a sequence associated with the correspondingly targeted gene.

8. The method of claim 1, wherein the methylation level of the STRIP1 gene is calculated using formula 2^{[Ct(β-actin)-Ct(STRIP1)]}×100, the methylation level of the PGAM2 gene is calculated using formula 2^{[Ct(β-actin)-Ct(PGAM2)]}×100, the methylation level of the AAMP gene is calculated using formula 2^{[Ct(β-actin)-Ct(AAMP)]}×100, and the methylation level of the ZNF497 gene is calculated using formula 2^{[Ct(β-actin)-Ct(ZNF497)]}×100.

9. A kit used in the method of claim 1, comprising:
a primer-pair and a probe for detecting the methylation level of the STRIP1 gene;
a primer-pair and a probe for detecting the methylation level of the ZNF649 gene;
a primer-pair and a probe for detecting the methylation level of the CFAP57 gene;
a primer-pair and a probe for detecting the methylation level of the GGTA1 gene;
a primer-pair and a probe for detecting the methylation level of the PGAM2 gene;
a primer-pair and a probe for detecting the methylation level of the FRMD4A gene;
a primer-pair and a probe for detecting the methylation level of the GJB6 gene;
a primer-pair and a probe for detecting the methylation level of the AAMP gene;
a primer-pair and a probe for detecting the methylation level of the miR-203A;
a primer-pair and a probe for detecting the methylation level of the SLC22A2 gene;
a primer-pair and a probe for detecting the methylation level of the BCAS3 gene;
a primer-pair and a probe for detecting the methylation level of the TSPYL5 gene; and
a primer-pair and a probe for detecting the methylation level of the ZNF497 gene.

10. A kit used in the method of claim 1, comprising:
a primer-pair and a probe for detecting the methylation level of the STRIP1 gene;
a primer-pair and a probe for detecting the methylation level of the PGAM2 gene;
a primer-pair and a probe for detecting the methylation level of the AAMP gene; and
a primer-pair and a probe for detecting the methylation level of the ZNF497 gene.


SEQUENCE LISTING (42 sequences)

SEQ ID NO: 1 - DNA, 22 bp, synthetic construct
  misc_feature 1..22: note = sense primer
  source 1..22: mol_type = other DNA
ggtagygttcggtcgakaattg

SEQ ID NO: 2 - DNA, 21 bp, synthetic construct
  misc_feature 1..21: note = antisense primer
  source 1..21: mol_type = other DNA
ctcccgaacgycgaaartaaa

SEQ ID NO: 3 - DNA, 25 bp, synthetic construct
  misc_feature 1..25: note = probe
  source 1..25: mol_type = other DNA
akgagtaggaagcgasgttggtagt

SEQ ID NO: 4 - DNA, 20 bp, synthetic construct
  misc_feature 1..20: note = sense primer
  source 1..20: mol_type = other DNA
ggaatcsgaagcgagsttta

SEQ ID NO: 5 - DNA, 20 bp, synthetic construct
  misc_feature 1..20: note = antisense primer
  source 1..20: mol_type = other DNA
accaatccraacgaccatrc

SEQ ID NO: 6 - DNA, 25 bp, synthetic construct
  misc_feature 1..25: note = probe
  source 1..25: mol_type = other DNA
mgtgagattkgttattattcggcgt

SEQ ID NO: 7 - DNA, 20 bp, synthetic construct
  misc_feature 1..20: note = sense primer
  source 1..20: mol_type = other DNA
gggtcgtttcgtasggattr

SEQ ID NO: 8 - DNA, 22 bp, synthetic construct
  misc_feature 1..22: note = antisense primer
  source 1..22: mol_type = other DNA
ctcstccttcacstcattacac

SEQ ID NO: 9 - DNA, 24 bp, synthetic construct
  misc_feature 1..24: note = probe
  source 1..24: mol_type = other DNA
aagtattgcggrttgggtttggcg

SEQ ID NO: 10 - DNA, 21 bp, synthetic construct
  misc_feature 1..21: note = sense primer
  source 1..21: mol_type = other DNA
ggttgaggtagkcggattatt

SEQ ID NO: 11 - DNA, 22 bp, synthetic construct
  misc_feature 1..22: note = antisense primer
  source 1..22: mol_type = other DNA
ctcaycctcccacgtaactyaa

SEQ ID NO: 12 - DNA, 24 bp, synthetic construct
  misc_feature 1..24: note = probe
  source 1..24: mol_type = other DNA
aattagttkggcgtggtggtycgt

SEQ ID NO: 13 - DNA, 22 bp, synthetic construct
  misc_feature 1..22: note = sense primer
  source 1..22: mol_type = other DNA
tttggacggtacygattagatg

SEQ ID NO: 14 - DNA, 21 bp, synthetic construct
  misc_feature 1..21: note = antisense primer
  source 1..21: mol_type = other DNA
ccgtacttaacgacmgtttct

SEQ ID NO: 15 - DNA, 24 bp, synthetic construct
  misc_feature 1..24: note = probe
  source 1..24: mol_type = other DNA
tgttrgtggtgcgtatttggcgtt

SEQ ID NO: 16 - DNA, 22 bp, synthetic construct
  misc_feature 1..22: note = sense primer
  source 1..22: mol_type = other DNA
ggmgagagaggtagagtgtgtt

SEQ ID NO: 17 - DNA, 22 bp, synthetic construct
  misc_feature 1..22: note = antisense primer
  source 1..22: mol_type = other DNA
aackaactcaactcccgataaa

SEQ ID NO: 18 - DNA, 28 bp, synthetic construct
  misc_feature 1..28: note = probe
  source 1..28: mol_type = other DNA
tgcggcgtatargtggtatagttaggga

SEQ ID NO: 19 - DNA, 23 bp, synthetic construct
  misc_feature 1..23: note = sense primer
  source 1..23: mol_type = other DNA
ggtatcggattagagtcggtmga

SEQ ID NO: 20 - DNA, 20 bp, synthetic construct
  misc_feature 1..20: note = antisense primer
  source 1..20: mol_type = other DNA
rgaaaccgaaacgcgtaaac

SEQ ID NO: 21 - DNA, 23 bp, synthetic construct
  misc_feature 1..23: note = probe
  source 1..23: mol_type = other DNA
tacgaccaaacacccatcccgyc

SEQ ID NO: 22 - DNA, 21 bp, synthetic construct
  misc_feature 1..21: note = sense primer
  source 1..21: mol_type = other DNA
tgaggamgaagaggaggaaga

SEQ ID NO: 23 - DNA, 22 bp, synthetic construct
  misc_feature 1..22: note = antisense primer
  source 1..22: mol_type = other DNA
ctatcgtcgaaacsctccatac

SEQ ID NO: 24 - DNA, 23 bp, synthetic construct
  misc_feature 1..23: note = probe
  source 1..23: mol_type = other DNA
aaycccaaccctcttcgttaccc

SEQ ID NO: 25 - DNA, 24 bp, synthetic construct
  misc_feature 1..24: note = sense primer
  source 1..24: mol_type = other DNA
cscggtttggagttagagttatag

SEQ ID NO: 26 - DNA, 20 bp, synthetic construct
  misc_feature 1..20: note = antisense primer
  source 1..20: mol_type = other DNA
tacgaaacycgacgcataaa

SEQ ID NO: 27 - DNA, 24 bp, synthetic construct
  misc_feature 1..24: note = probe
  source 1..24: mol_type = other DNA
acgcgaaacmgatccgtaaactcc

SEQ ID NO: 28 - DNA, 25 bp, synthetic construct
  misc_feature 1..25: note = sense primer
  source 1..25: mol_type = other DNA
gggtgattatcgttgttatrgaagg

SEQ ID NO: 29 - DNA, 23 bp, synthetic construct
  misc_feature 1..23: note = antisense primer
  source 1..23: mol_type = other DNA
aatttcccacataacctctacsc

SEQ ID NO: 30 - DNA, 26 bp, synthetic construct
  misc_feature 1..26: note = probe
  source 1..26: mol_type = other DNA
tsgagaatgtcgatgatgggtagtgg

SEQ ID NO: 31 - DNA, 22 bp, synthetic construct
  misc_feature 1..22: note = sense primer
  source 1..22: mol_type = other DNA
ggtatggtggtacgtgttrgta

SEQ ID NO: 32 - DNA, 23 bp, synthetic construct
  misc_feature 1..23: note = antisense primer
  source 1..23: mol_type = other DNA
ctcgctctatcacccaawctaaa

SEQ ID NO: 33 - DNA, 26 bp, synthetic construct
  misc_feature 1..26: note = probe
  source 1..26: mol_type = other DNA
tcgggaggtagaggtrgtagtgagtt

SEQ ID NO: 34 - DNA, 22 bp, synthetic construct
  misc_feature 1..22: note = sense primer
  source 1..22: mol_type = other DNA
ggagsgtttrgtcgtmgatatt

SEQ ID NO: 35 - DNA, 18 bp, synthetic construct
  misc_feature 1..18: note = antisense primer
  source 1..18: mol_type = other DNA
aaaccacsccgatttcsa

SEQ ID NO: 36 - DNA, 24 bp, synthetic construct
  misc_feature 1..24: note = probe
  source 1..24: mol_type = other DNA
ttkcgtgggaatagcgsgaatmgt

SEQ ID NO: 37 - DNA, 19 bp, synthetic construct
  misc_feature 1..19: note = sense primer
  source 1..19: mol_type = other DNA
cgaggcgggrggattattt

SEQ ID NO: 38 - DNA, 23 bp, synthetic construct
  misc_feature 1..23: note = antisense primer
  source 1..23: mol_type = other DNA
tckcgatctcctaacctcgtaat

SEQ ID NO: 39 - DNA, 24 bp, synthetic construct
  misc_feature 1..24: note = probe
  source 1..24: mol_type = other DNA
tttatcggtcgsgtgtcgtggttt

SEQ ID NO: 40 - DNA, 27 bp, synthetic construct
  misc_feature 1..27: note = sense primer
  source 1..27: mol_type = other DNA
ggtggaggtagttagggtttatttgta

SEQ ID NO: 41 - DNA, 31 bp, synthetic construct
  misc_feature 1..31: note = antisense primer
  source 1..31: mol_type = other DNA
ccacaccacaaaatcacacttaacctcattt

SEQ ID NO: 42 - DNA, 21 bp, synthetic construct
  misc_feature 1..21: note = probe
  source 1..21: mol_type = other DNA
cacttttattcaactaatctc